(19) United States
(12) Patent Application Publication
(10) Pub. No.: US 2008/0112842 A1
(43) Pub. Date: May 15, 2008

(54) MONITOR DRAPE WITH VENTS

(75) Inventor: Craig Edwards, Mission Viejo, CA (US)

Correspondence Address: ADVANCED MEDICAL OPTICS, INC., 1700 E. ST. ANDREW PLACE, SANTA ANA, CA, 92705, US

(73) Assignee: Advanced Medical Optics, Inc., Santa Ana, CA (US)

(21) Appl. No.: 11/558,431

(22) Filed: Nov. 9, 2006

(51) Int. Cl. A61L 2/26; B65D 65/18; B65D 65/06

(52) U.S. Cl. 422/2; 422/40; 150/154

(57) ABSTRACT

A sterile drape maintains a sterile field during use of a flat panel touch screen during diagnostic and/or therapeutic procedures. The drape comprises sterile front and rear sheets formed from a thin layer of a transparent material that accommodates the transmission of tactile pressure. The rear sheet may comprise a folded extension of the front sheet along the top edge, with the sheets bonded along the sizes. A bottom opening between the front and the rear sheets allows cooling air to enter, and at least one ventilation opening is near the top.

### BACKGROUND OF THE INVENTION

The present invention relates to devices, methods, and systems used in surgery. Exemplary embodiments specifically relate to the maintenance of a sterile field in the area around the surgical procedure during treatments and/or measurements of the eye.

Surgical and diagnostic procedures involving the tissues of the eye often employ sophisticated surgical systems or devices. To facilitate accurate measurements and therapies to the tissues of the eye, these system often display information and receive inputs while the system is in use for a particular patient. Flat panel display screens are being used with greater and greater frequency to display information to the surgical personnel, and in many cases, to receive input or commands from the surgeon (or other medical professionals) during medical procedures through the use of flat panel touch screen structures.

More generally, touch panel display screens are often placed in or near the sterile field for use in preparation for, during, and/or after a wide variety of surgical or diagnostic procedures. Since complex display screens and computerized systems may not be easily sterilized, it can be advantageous to place a sterile drape over the display screen. However, existing sterile drapes may not be optimized for use with surgical touch screen displays. For example some existing sterile drapes may hinder the display of precise images and graphic information, and/or may hinder inputting of commands to the touch screen. Other sterile drapes may not be easily and effectively positioned for surgery or readily removed after surgery, and they may not fit a wide range of display screen structures. The difficulty in accurate fitting of sterile drapes to flat panel displays may result, for example, in either bunching of the drape material (when elastic or tape is used to allow a drape to be used with a range of different display sizes) or excessive surgical drape inventory costs and complexity (when a large number of different specialized drapes are needed for a number of different displays).

Accordingly, it would be desirable to provide a sterile drape optimized for use with flat panel display screens and touch screen displays. It would be further desirable to provide a drape having a high transparency and which allows an accurate transmittance of tactile pressure, but which can be easily deployed on to and removed off of a flat panel touch screen, ideally without bunching of the material, without relying on repeated application of adhesives onto the display surface (which might otherwise build-up and decrease display resolution), and without other deleterious effects on the life or functioning of a surgical touch screen display, all while maintaining patient safety and the appropriate sterile field.

### BRIEF SUMMARY OF THE INVENTION

The present invention generally provides improved sterile drapes for flat panel displays, and often for flat panel touch screen displays. Embodiments of the invention may allow a sterile field to be easily established before use, and may maintain the sterile field during use of a touch screen user interface by surgical personnel during surgery. Advantageously, a simple folded sheet of a suitable transparent sterile material can be fused along its sides, with a front sheet portion of the material being allowed to hang freely along the display surface. The rear sheet can be shorter than the front, and the drape may be open at the bottom with ventilation holes along the top. This simple open arrangement may avoid wrinkles or bunching of the material along the display (which might otherwise result from the use of adhesives, tape, or elastic) and may allow the user to view and actuate a touch screen user interface while accommodating convective cooling of the flat panel display. After use, the drape may be readily lifted off the screen, inhibiting any inadvertent yanking on the display screen or its support structure.

In one aspect, the present invention provides a sterile drape for a flat panel display having a display surface. The sterile drape comprises a sterile front sheet at least a large as the display surface, the front sheet comprising a thin layer of a transparent material and having a top edge and a bottom edge. A rear sheet has at least one opening for ventilation, and the rear sheet is attached to the front sheet near the top edge. A rear bottom edge of the rear sheet is detached from the front sheet so as to form a hood with an open bottom, the front sheet and the rear sheet configured for draping over the flat panel display so as to maintain a sterile field adjacent the display surface.

Typically, the front sheet is substantially rectangular in shape, and a plurality of openings may be distributed across the rear sheet near the top edge. The openings may be disposed along the top and/or rear surface of the flat panel display when the sterile drape is positioned for use. The rear sheet can be significantly shorter than a height of the front sheet between the top edge and the bottom edge so as to leave a lower rear portion of the flat panel display uncovered. This can help to accommodate support structures associated with the display without delaying surgical preparations for draping structures that the surgical staff will not touch and/or contaminate during normal surgical system use.

The rear sheet may be formed from a folded extension of the front sheet, with the left and right edges of the front and rear sheets being fused, adhesively bonded, fastened, or otherwise affixed together. The front sheet and the rear sheet will typically comprise a thin layer of flexible polymer material suitable for viewing the display surface therethrough, with the layer often being configured to accommodate the transmission of tactile touch-screen inputs from the sterile field to the flat panel display while maintaining the sterile field. The bottom opening between the front sheet and the rear sheet, together with a plurality of openings distributed between left and right edges near the top edge, may allow sufficient air heated by the flat panel display to be ventilated that the entire flat panel display is cooled. A single fold may separate the front and rear sheets along their entire width, although more complex fold and/or seam arrangements may also be included, optionally with the rear sheet having a top portion extending along a top surface of the display. Nonetheless, the rear sheet generally will extend downward and/or rearward from the top edge of the front sheet.

In another aspect, the invention provides a sterile drape for maintaining a sterile field adjacent a flat panel touch-screen display. The flat panel display has a display surface between top, bottom, left, and right side edges, along with a thickness between a back surface and the display surface. The sterile drape comprises a sterile front sheet comprising a thin layer of a transparent material having a top edge, a bottom edge, a left edge, and a right edge. A rear sheet (formed by a folded extension of the thin transparent material from the front sheet) extends from the top edge of the front sheet, the rear sheet being fused to the front sheet along the left and right edges. The front sheet and rear sheet having a bottom opening between them, and the bottom opening the flat panel display can slide into that opening so that the front sheet remains aligned with the display surface when touch commands are transmitted from a user in the sterile field thereto. The bottom remains open when the flat panel display is between the front and rear sheets, and at least one upper opening in the thin transparent material near the top edge allows ventilation to flow in through the bottom opening and then out.

### BRIEF DESCRIPTION OF THE DRAWINGS

FIG. 1 is a schematic illustration of an eye surgery system having a user interface making use of a flat panel display touch screen, in which an embodiment of a surgical drape of the present invention is disposed over the touch screen display surface of the flat panel.

FIG. 2A is a front perspective view of a flat panel touch screen display with an exemplary embodiment of a surgical drape of the present invention disposed thereon.

FIG. 2B is a rear perspective view of the flat panel touch screen display and surgical drape of FIG. 2A.

FIG. 3 is an alternative rear perspective of the surgical drape of FIG. 2A.

FIG. 4 is a rear view of the surgical drape of FIG. 2A, schematically showing how the rear sheet can be formed from an extension of the front sheet material by folding the material along the top edge.

### DETAILED DESCRIPTION OF THE INVENTION

According to embodiments of the present invention, a sterile drape for use during surgical procedures is provided. More specifically, the present invention relates to a sterile drape used to maintain a sterile field along a touch screen, such as those used with a variety of surgical systems and devices. By way of example, the invention can be applied to eye treatment systems such as phacoemulsification system for treatment of tissues within the eye, laser vision correction systems, and the like, generally to maintain a sterile field along flat panel display and/or touch screens.

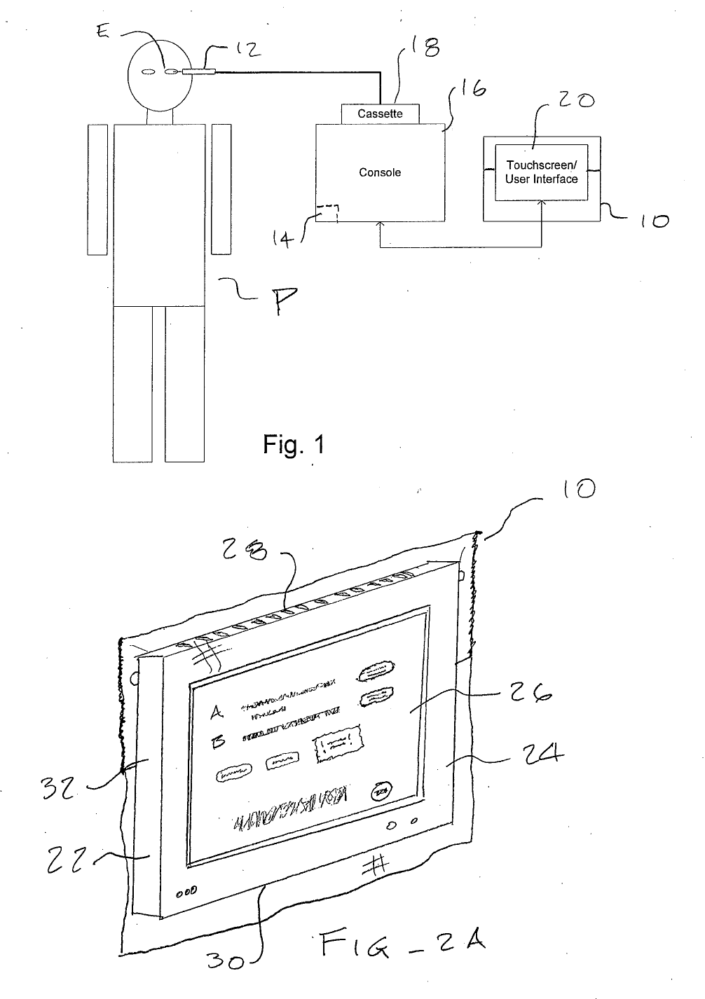

One embodiment of a surgical drape 10 used with an eye surgery system is shown schematically in FIG. 1. A probe handpiece 12 is used by a surgeon to treat an eye E of a patient P. The probe here may comprise, for example, a phacoemulsification probe used to treat cataracts of the eye E. A tip of probe 12 is inserted into the eye and energized to transmit ultrasound energy that breaks up tissues, and simultaneous aspiration and irrigation fluid flows are directed through the probe tip to remove the tissues while maintaining the internal eye fluid volume and pressure.

The operation of probe 12 is controlled by a processor 14 of a surgical console per the commands of the surgeon. A cassette 18 couples the probe 12 to the console 16, and the surgeon monitors the status of the surgical system and inputs commands to the surgical system at least in part using a touch screen user interface 20 via a flat panel touch screen display. Surgical drape 10 extends over the touch screen display of the user interface, allowing the surgeon to see the information presented by the display and to transmit commands to processor 14 by touching the display screen with a finger, a tool, and/or the like. Suitable eye surgery systems for use with surgical drape 10 include those commercially available from Advanced Medical Optics, Inc. of Santa Ana, Calif.; Alcon Laboratories of Fort Worth, Tex., Bausch and Lomb of Rochester, N.Y., and others. A wide range of alternative eye treatment and/or measurement systems and devices might also employ similar surgical drapes, including the laser vision colTection systems and/or wavefront measurement systems commercially available from VISX of Santa Clara, Calif. and others. Embodiments of the surgical drapes of the present invention may also find use with a wide variety of alternative medical treatment and/or diagnostic systems used to address disease states of a wide variety of tissues in the body in a range of different clinical and research settings.

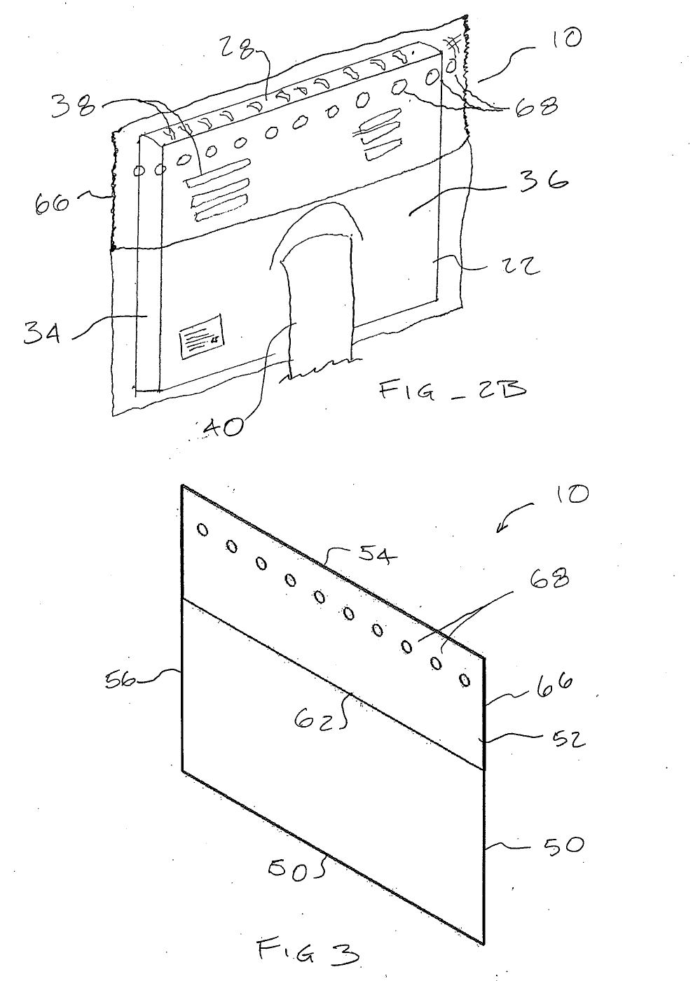

Referring now to FIGS. 2A and 2B, surgical drape 10 is seen disposed over a flat panel display 22. Flat panel 22 generally includes a front 24 having a touch screen display surface 26. Flat panel 22 generally includes a top 28 and bottom 30 surfaces, with a height therebetween. Left 32 and right 34 surfaces of flat panel generally define its width, and the height and width of the flat panel are typically greater than a thickness between front 24 and a rear surface 36. Vents 38 may be formed in the top 28, bottom 30, left 32, right, 34, and/or rear 36 surfaces of the flat panel to allow cooling of the display, electronics, and the like, and vents might also be found in front surface 24 outside of display 26 in some embodiments. A support structure 40 extends from a lower portion of rear surface 36. Exemplary touch screen flat panel displays suitable for use in surgical systems include those commercially available from L.G. Phillips located in Korea, and the like.

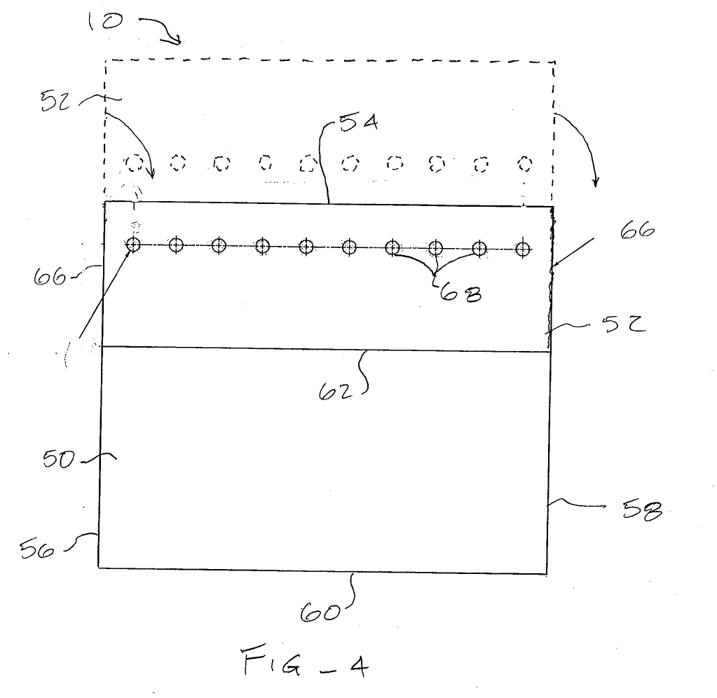

Referring now to FIGS. 3 and 4, surgical drape 10 generally comprises a front sheet 50 and a rear sheet 52, both formed of a thin layer of transparent material. The front and rear sheets may be of a generally rectangular shape corresponding to (though often larger and/or taller in height than) the shape of the flat panel display and/or the display screen, although other suitable shapes might also be used. Regardless, surgical drape 10 will typically include a top edge 54, a right edge 56, and a left edge 58, with the front and rear sheets 50, 52 each having an associated bottom edge 60, 62, respectively. Bottom edge 60 of front sheet 50 will often extend below bottom edge 62 to cover display 26 while leaving a lower portion of the rear surface 36 of the flat panel uncovered. Alternatively, the bottom edge 62 may extend to or below the bottom edge 60 of the front sheet 50.

The transparent material from which front sheet 50 and rear sheet 52 are formed generally allows easy viewing of the information displayed on the flat panel touch screen. The thin layer accommodates the transmission of tactile pressure from the sterile field to the touch screen for providing touch commands. The thin layer may also be of sufficient flexibility so that the front sheet substantially conforms to the display surface of the flat panel touch screen. Substantially conforming to the front sheet of the display surface may advantageously reduce optical distortions and ease the operation of the touch screen. The material may be of a suitable polymer, for example, a high clarity, low density polyethylene resin such as that commercially available from Dow Chemical under model number 503A, Certene™ LDF 221 from Muehlstein, Chevron Phillips 5355, and/or the like. Sheets 50 and 52 may have a thickness in a range from about 1 mil to about 10 mil, with an exemplary embodiment having a thickness of about 2 mil to about 4 mil. In some embodiments, the rear sheet may be formed of a different and/or even non-transparent material, since the rear sheet does not cover the display portion of the display screen.

As can be understood with reference to FIG. 4, rear sheet 52 may be formed by folding an extension of the material of front sheet 50 along top edge 54, so that the top edge of surgical drape comprises a folded edge. Side weld heatseals 66 may optionally be formed between the adjacent right edges of front sheet 50 and rear sheet 52, and also between the adjacent left edges. More generally, as can be seen in FIGS. 2A an 2B, the rear sheet is attached to the front sheet so as to form a hood. When the drape is placed over a flat panel touch screen, the hood slides over the top of the flat panel touch screen to hold the drape in place. An opening at the bottom of the front and the rear sheet facilitates easily sliding the drape on and off the flat panel touch display, and also allows air to circulate from beneath the drape. The flexible layers of the sheets also ease placing and removing the drape.

Surgical drape 10 includes ventilation openings 68 to allow air heated by the flat panel touch screen to escape. Cooled air is often drawn in through the bottom opening. The ventilation openings 68 may be of a suitable shape and number to allow the desired cooling while maintaining the sterile field along display 26. The ventilation openings may be distributed sufficiently evenly across the hood so as to facilitate ventilation of substantially the entire flat panel touch screen, with exemplary embodiments including between about 5 and 50 openings disposed in a single row near top edge 54, with each opening being between about 0.25 inches and about 0.75 inches in diameter. The openings 68 may be circular, as I the illustrated embodiment, or may gave some other shape appropriate for ventilation, for example, rectangular, square, round, oval, and/or generally elongated. In certain embodiments, the openings may have a predetermined relationship with at least some of the vents 38, for example, the same spacing, a similar size, and/or a similar shape to the vents 30.

The drape may be pre-packaged in a sterilized package, and may be used in a single-use manner (with disposal after use for a single patient). This obviates any necessity to clean and sterilize the drape after use.

While the above is a complete description of the preferred embodiments of the invention, various alternatives, modifications, and equivalents may be used. Therefore, the above description should not be taken as limiting the scope of the invention which is defined by the appended claims.

What is claimed is:

1. A sterile drape for a flat panel display having a display surface, comprising:
a sterile front sheet at least a large as the display surface, the front sheet comprising a thin layer of a transparent material and having a top edge and a bottom edge; and
a rear sheet having at least one opening for ventilation, the rear sheet attached to the front sheet near the top edge and having rear bottom edge detached from the front sheet so as to form a hood with an open bottom, the front sheet and the rear sheet configured for draping over the flat panel display so as to maintain a sterile field adjacent the display surface.

2. The sterile drape of claim 1, wherein the front sheet is substantially rectangular in shape, and wherein the rear sheet has a plurality of openings distributed across the rear sheet near the top edge.

3. The sterile drape of claim 2, wherein the rear sheet is substantially rectangular in shape with a rear height between the top edge an the rear bottom edge being significantly less than a height of the front sheet between the top edge and the bottom edge so as to leave a lower rear portion of the flat panel display uncovered.

4. The sterile drape of claim 1, wherein the rear sheet comprises a folded extension of the front sheet, the rear sheet having left and right edges affixed to left and right edges of the front sheet.

5. The sterile drape of claim 4, wherein the front sheet and the rear sheet comprise a thin layer of flexible polymer material suitable for viewing the display surface therethrough.

6. The sterile drape of claim 1, wherein the thin layer is configured to accommodate the transmission of tactile touch-screen inputs from the sterile field to the flat panel display while maintaining the sterile field.

7. The sterile drape of claim 1, wherein the sterile drape has a bottom opening between the front sheet and the rear sheet corresponding to a bottom portion of the flat panel display when the sterile drape is positioned thereon, and wherein the at least one opening comprises a plurality of openings distributed between left and right edges of the rear sheet sufficiently near the top edge to allow air heated by the flat panel display to enter the bottom opening and exit the plurality of openings such that substantially the entire flat panel display is ventilated and cooled.

8. A sterile drape for maintaining a sterile field adjacent a flat panel touch-screen display, the flat panel display having a display surface between top, bottom, left, and right side edges, the flat panel display having a thickness between a back surface and the display surface, the sterile drape comprising:
a sterile front sheet comprising a thin layer of a transparent material and having a top edge, a bottom edge, a left edge, and a right edge;
a rear sheet comprising a folded extension of the thin transparent material of the front sheet extending from the top edge of the front sheet, the rear sheet fused to the front sheet along the left edge and along the right edge, the front sheet and rear sheet having a bottom opening therebetween, the bottom opening slidably receiving the flat panel display so that the front sheet remains aligned with the display surface when touch commands are transmitted from a user in the sterile field thereto and the bottom remains open; and
at least one upper opening in the thin transparent material near the top edge for ventilation to flow in through the bottom opening and out through the at least one opening during use.
